(19) United States
(12) Patent Application Publication
(10) Pub. No.: US 2007/0042427 A1
(43) Pub. Date: Feb. 22, 2007

(54) Microfluidic laminar flow detection strip

(75) Inventors: John Gerdes, Columbine Valley, CO (US); C. Frederick Battrell, Redmond, WA (US); Denise Maxine Hoekstra, Monroe, WA (US); John Clemmens, Redmond, WA (US); Stephen Mordue, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Micronics, Inc., Redmond, WA (US)

(21) Appl. No.: 11/416,791

(22) Filed: May 3, 2006

Related U.S. Application Data
(60) Provisional application No. 60/677,531, filed on May 3, 2005.

(51) Int. Cl. G01N 33/53; C12M 3/00

(52) U.S. Cl. 435/7.1; 435/287.2

(57) ABSTRACT

The present invention relates to microfluidic laminar flow detection strip devices and methods for using and making the same. The disclosed devices comprise: a first inlet; a microfluidic channel having a first end and a second end, wherein the first end is fluidly connected to the first inlet; a bellows pump fluidly connected to the second end of the microfluidic channel, wherein the bellows pump comprises an absorbent material disposed therein; a dried reagent zone within the microfluidic channel, wherein the dried reagent zone comprises a first reagent and a control reagent printed thereon, the first reagent comprising a first detection antibody conjugated to a dyed substrate bead or functionalized for colorimetric development, and the control reagent comprising a control detection antibody conjugated to a dyed substrate bead or functionalized for calorimetric development; a first bound antibody zone within the microfluidic channel, wherein the first bound antibody zone comprises a first bound antibody printed thereon; and a control zone within the microfluidic channel, wherein the control zone comprises a control bound antibody printed thereon.

### CROSS-REFERENCE TO RELATED APPLICATION

This application claims the benefit under 35 U.S.C. §119(e) of U.S. Provisional Patent Application No. 60/677,531, filed May 3, 2005, where this provisional application is incorporated herein by reference in its entirety.

### BACKGROUND OF THE INVENTION

1. Field of the Invention

The present invention relates generally to microfluidic devices, and, more particularly, to microfluidic laminar flow detection strip devices and methods for using and making the same.

2. Description of the Related Art

Detection of biological or chemical analytes in point-of-care or field testing environments (such as a doctor's office, food or water processing plant, or home setting) offers significant advantages, including obtaining a more rapid result that enables immediate on site intervention based upon the test. However, such environments require that the detection methods be of low cost and simple assay complexity. Preferably, the detections methods would require no instrumentation for sample processing or result interpretation.

Immunochromatographic tests, referred to as lateral flow (LF) tests have been widely used for qualitative and semi-quantitative assays relying on visual detection. One advantage to these types of tests is that execution typically does not require additional specialized equipment or trained personnel. Another advantage is the wide variety of analytes that can be detected using this type of test. Consequently, a large industry exists for commercialization of this methodology. See, e.g., U.S. Pat. No. 5,120,643, U.S. Pat. No. 4,943,522, U.S. Pat. No. 5,770,460, U.S. Pat. No. 5,798,273, U.S. Pat. No. 5,504,013, U.S. Pat. No. 6,399,398, U.S. Pat. No. 5,275,785, U.S. Pat. No. 5,504,013, U.S. Pat. No. 5,602,040, U.S. Pat. No. 5,622,871, U.S. Pat. No. 5,656,503, U.S. Pat. No. 4,855,240, U.S. Pat. No. 5,591,645, U.S. Pat. No. 4,956,302, U.S. Pat. No. 5,075,078, and U.S. Pat. No. 6,368,876.

Although lateral flow assays have been developed extensively for detection of antigens or antibodies, the application of such assays to nucleic acid detection has yet to be fully developed. Oligonucleotide probes are increasingly being utilized in diagnostics since they can be arrayed for detection of multiple analytes and can provide much greater assay sensitivity and specificity, especially when combined with isothermal or PCR-based amplification methods. See, e.g., U.S. Pat. No. 5,981,171, U.S. Pat. No. 5,869,252, U.S. Pat. No. 6,210,898, U.S. Pat. No. 6,100,099, and U.S. Patent Application Publication No. 2004/0110167.

Although conventional rapid lateral flow assays that utilize porous membranes are a popular choice for determining the presence of a given analyte in a sample, they are not without their shortcomings. Most importantly, the sensitivity of such assays has often been questioned due to various limitations associated with the currently available formats (see, e.g., Giles et al., Journal of Medical Virology 59:104-109 (1999)). Other practical limitations to the use of these assays is inherent in the use of a membrane in the design of the assay. For example, a membrane can become “plugged” when utilizing complex biological sample, such as blood or culture fluids. In some instances, flow through or wash steps could provide a means for the removal of background materials, such as cells or other matrix substances, that might plug the membrane. However, the lateral flow format does not allow for a washing step due to the membrane flow-through format. Accordingly, any interfering species, such as particulate or colored material introduced by the sample solution, or unbound label, can potentially interfere with the readout of the assay device. One solution that has been investigated is a lateral flow format employing filtration during the assay procedure, e.g., using specially coated filters to remove potential interfering species prior to detection of the analyte (see, e.g., U.S. Pat. No. 4,933,092, U.S. Pat. No. 5,452,716, and U.S. Pat. No. 5,665,238).

It is well known that flow rate and adequate contact between the analyte and its corresponding capture antibody immobilized within the membrane are critical to the assay sensitivity. This demands careful membrane selection to optimize dwell time and flow rates. Significant improvements could be made if these parameters could be more conveniently controlled and optimized. For example, U.S. Pat. No. 6,849,414 describes a lateral flow assay featuring the controlled release of reagents that achieves greater sensitivity than conventional rapid test assays. In alternate example, the membrane is eliminated and other means are used to control fluid flow (see, e.g., U.S. Pat. No. 5,885,527, U.S. Patent Application Publication No. 2005/0014246, and U.S. Patent Application No. 2003/0129671). However, such systems typically rely on external pumps to regulate flow.

Although there have been many advances in the field, there remains a need for new and improved devices for detecting biological and chemical analytes in point-of-care or field testing environments. The present invention addresses these needs and provides further related advantages.

### BRIEF SUMMARY OF THE INVENTION

In brief, the present invention relates to microfluidic laminar flow detection strip devices and methods for using and making the same.

In one embodiment, a microfluidic laminar flow detection strip device is provided that comprises: (a) a first inlet; (b) a microfluidic channel having a first end and a second end, wherein the first end is fluidly connected to the first inlet; (c) a bellows pump fluidly connected to the second end of the microfluidic channel, wherein the bellows pump comprises an absorbent material disposed therein; (d) a dried reagent zone within the microfluidic channel, wherein the dried reagent zone comprises a first reagent and a control reagent printed thereon, the first reagent comprising a first detection antibody conjugated to a dyed substrate bead or functionalized for colorimetric development, and the control reagent comprising a control detection antibody conjugated to a dyed substrate bead or functionalized for calorimetric development; (e) a first bound antibody zone within the microfluidic channel, wherein the first bound antibody zone comprises a first bound antibody printed thereon; and (f) a control zone within the microfluidic channel, wherein the control zone comprises a control bound antibody printed thereon.

In a further embodiment, the device further comprises a second inlet fluidly connected to the first end of the microfluidic channel.

In another further embodiment, the dried reagent zone further comprises a second reagent printed thereon, and the second reagent comprises a second detection antibody conjugated to a dyed substrate bead or functionalized for colorimetric development; and the device further comprises a second bound antibody zone within the microfluidic channel, wherein the second bound antibody zone comprises a second bound antibody printed thereon.

In another further embodiment, the dried reagent zone further comprises a third reagent printed thereon, and the third reagent comprises a third detection antibody conjugated to a dyed substrate bead or functionalized for colorimetric development; and the device further comprises a third bound antibody zone within the microfluidic channel, wherein the third bound antibody zone comprises a third bound antibody printed thereon.

In another further embodiment, the bellows pump further comprises a vent hole.

In another further embodiment, the device further comprises: (a) a first check valve fluidly connected to the bellows pump, wherein the first check valve permits fluid flow from the microfluidic channel into the bellows pump and prevents fluid flow from the bellows pump into the microfluidic channel; and (b) a second check valve fluidly connected to the bellows pump, wherein the second check valve permits fluid flow away from the bellows pump.

In another further embodiment, the microfluidic channel has a serpentine shape.

In another further embodiment, the second end of the microfluidic channel is sized to control fluid flow rate within the microfluidic channel. More specifically, the second end of the microfluidic channel has a diameter of 25-500 μm, or, in more specific embodiments, 50-100 μm.

In another further embodiment, the device further comprises optical viewing windows positioned over the first bound antibody zone and the control zone. In certain embodiments, the optical viewing windows may be labeled

In certain embodiments, the first detection antibody is the same as the first bound antibody. In other embodiments, the first detection antibody is different than the first bound antibody. Similarly, in certain embodiments, the control detection antibody is the same as the control bound antibody. In other embodiments, the control detection antibody is different than the control bound antibody.

In certain embodiments, the device may be formed from a plurality of laminate layers. In other embodiments, the device may be formed from two injection molded layers and an adhesive layer.

In a second embodiment, a method of using the foregoing microfluidic laminar flow detection strip devices to detect the presence of an analyte of interest in a liquid sample is provided that comprises: (a) introducing the liquid sample into the first inlet of the device; (b) depressing the bellows pump; (c) releasing the bellows pump to draw the liquid sample through the microfluidic channel; and (d) visually inspecting the first bound antibody zone and the control zone for any color changes.

In a more specific embodiment of the foregoing method, the first reagent comprises a first detection antibody functionalized for calorimetric development; the control reagent comprises a control detection antibody functionalized for colorimetric development; and the method further comprises the following steps prior to the step of visually inspecting the first bound antibody zone and the control zone: (a) introducing a developing solution into the first inlet of the device; (b) depressing the bellows pump; and (c) releasing the bellows pump to draw the developing solution through the microfluidic channel.

These and other aspects of the invention will be apparent upon reference to the attached figures and following detailed description.

### BRIEF DESCRIPTION OF THE SEVERAL VIEWS OF THE DRAWINGS

FIGS. 1A-1F are a series of cross-sectional views illustrating the operation of a first embodiment of a microfluidic laminar flow detection strip device in accordance with aspects of the present invention.

FIGS. 2A-2F are a series of cross-sectional views illustrating the operation of a second embodiment of a microfluidic laminar flow detection strip device in accordance with aspects of the present invention.

FIGS. 3A-3F are a series of cross-sectional views illustrating the operation of a third embodiment of a microfluidic laminar flow detection strip device in accordance with aspects of the present invention.

FIGS. 4A-4H illustrate the individual laminate layers which are laminated together to form the microfluidic laminar flow detection strip device of FIGS. 3A-3F.

FIGS. 5A-5F are a series of cross-sectional views illustrating the operation of a fourth embodiment of a microfluidic laminar flow detection strip device in accordance with aspects of the present invention.

FIGS. 6A-6C illustrate the two injection molded layers and the adhesive layer which are assembled together to form the microfluidic device of FIGS. 1A-1F.

### DETAILED DESCRIPTION OF THE INVENTION

As noted previously, the present invention relates to microfluidic laminar flow detection strip devices and methods for using and making the same. The devices of the present invention utilize microfluidic channels, inlets, valves, pumps, liquid barriers and other elements arranged in various configurations to manipulate the flow of a liquid sample in order to qualitatively analyze the liquid sample for the presence of one or more analytes of interest. In the following description, certain specific embodiments of the present devices and methods are set forth, however, persons skilled in the art will understand that the various embodiments and elements described below may be combined or modified without deviating from the spirit and scope of the invention.

Microfluidic devices have become popular in recent years for performing analytical testing. Using tools developed by the semiconductor industry to miniaturize electronics, it has become possible to fabricate intricate fluid systems which can be analytical techniques for the acquisition and processing of information. The ability to perform analyses microfluidically provides substantial advantages of throughput, reagent consumption, and automatability. Another advantage of microfluidic systems is the ability to integrate a plurality of different operations in a single “lab-on-a-chip” device for performing processing of reactants for analysis and/or synthesis.

Microfluidic devices may be constructed in a multi-layer laminated structure wherein each layer has channels and structures fabricated from a laminate material to form microscale voids or channels where fluids flow. A microscale or microfluidic channel is generally defined as a fluid passage which has at least one internal cross-sectional dimension that is less than 500 μm and typically between about 0.1 μm and about 500 μm.

U.S. Pat. No. 5,716,852, which patent is hereby incorporated by reference in its entirety, is an example of a microfluidic device. The '852 patent teaches a microfluidic system for detecting the presence of analyte particles in a sample stream using a laminar flow channel having at least two input channels which provide an indicator stream and a sample stream, where the laminar flow channel has a depth sufficiently small to allow laminar flow of the streams and length sufficient to allow diffusion of particles of the analyte into the indicator stream to form a detection area, and having an outlet out of the channel to form a single mixed stream. This device, which is known as a T-Sensor, allows the movement of different fluidic layers next to each other within a channel without mixing other than by diffusion. A sample stream, such as whole blood, a receptor stream, such as an indicator solution, and a reference stream, which may be a known analyte standard, are introduced into a common microfluidic channel within the T-Sensor, and the streams flow next to each other until they exit the channel. Smaller particles, such as ions or small proteins, diffuse rapidly across the fluid boundaries, whereas larger molecules diffuse more slowly. Large particles, such as blood cells, show no significant diffusion within the time the two flow streams are in contact.

Typically, microfluidic systems require some type of external fluidic driver to function, such as piezoelectric pumps, micro-syringe pumps, electroosmotic pumps, and the like. However, in U.S. Pat. No. 6,743,399, which patent is hereby incorporated by reference in its entirety, microfluidic systems are described which are completely driven by inherently available internal forces such as gravity, hydrostatic pressure, capillary force, absorption by porous material or chemically induced pressures or vacuums.

In addition, many different types of valves for use in controlling fluids in microscale devices have been developed. For example, U.S. Pat. No. 6,432,212 describes one-way valves (also known as check valves) for use in laminated microfluidic structures, U.S. Pat. No. 6,581,899 describes ball bearing valves for use in laminated microfluidic structures, U.S. Patent Application Publication No. 2002/0148992, which application is assigned to the assignee of the present invention, describes a pneumatic valve interface, also known as a zero dead volume valve or passive valve, for use in laminated microfluidic structures, and U.S. Provisional Patent Application entitled “Electromagnetic Valve Interface for Use in Microfluidic Structures”, filed on Jan. 13, 2006 and assigned to the assignee of the present invention, describes an electromagnetically actuated valve interface for use in laminated microfluidic structures. The foregoing patents and patent applications are hereby incorporated by reference in their entirety.

As one of ordinary skill in the art will appreciate, the terms “analyte of interest” used herein includes (but is not limited to) analytes and antigens, such as proteins, peptides, nucleic acids, enzymes, hormones, therapeutic drugs, drugs of abuse, infection agents, biothreat agents, cells, cell organelles, or other compounds of interest in a sample.

In addition, as one of ordinary skill in the art will appreciate, the terms “liquid sample” and “biological sample” used herein includes (but is not limited to) liquid biological samples such as blood, plasma, serum, spinal fluid, saliva, urine, stool, and semen samples. In addition, as one of ordinary skill in the art will appreciate, such liquid biological samples may be subject to pre-processing steps, such as separation, filtration, purification and centrifugation/phase separation steps.

In addition, as one of ordinary skill in the art will appreciate “detection” may occur by any number of alternative methods. In the following description, and illustrated embodiments, detection occurs via visual detection using captured dyed conjugated microparticles or colorimetric development. However, other detection methods, such as fluorescent nanocrystals, Ramen scattering, direct fluorescence, or chemoluminescence, may be utilized through the incorporation of an appropriate signal detection device.

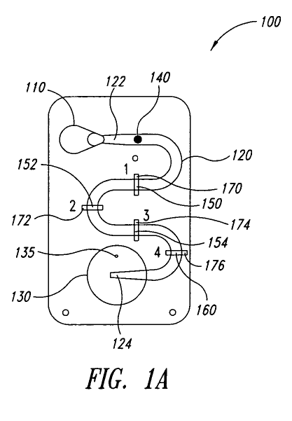

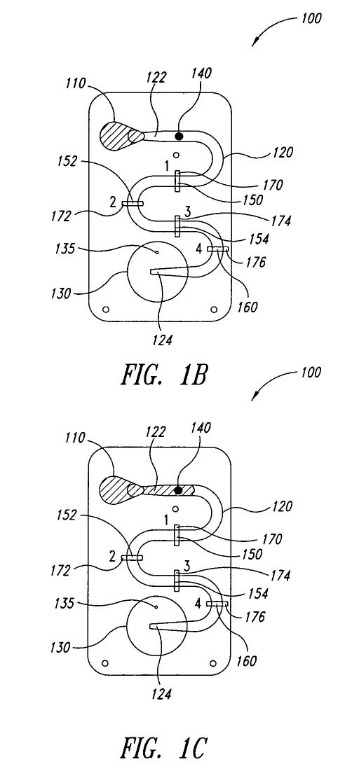

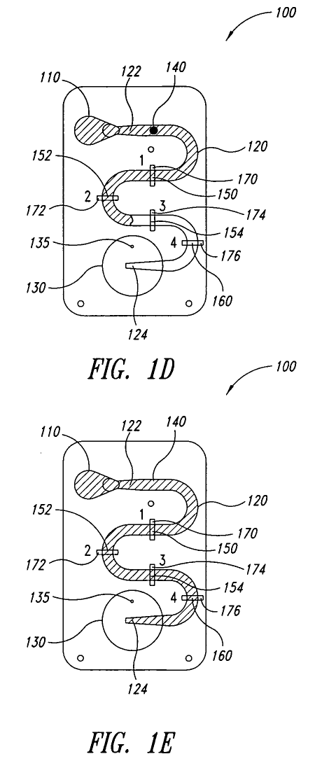

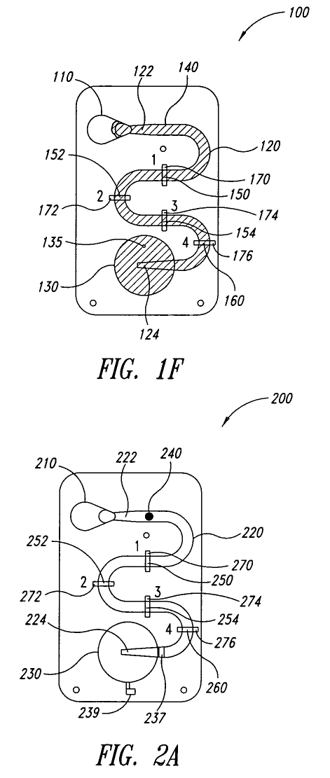

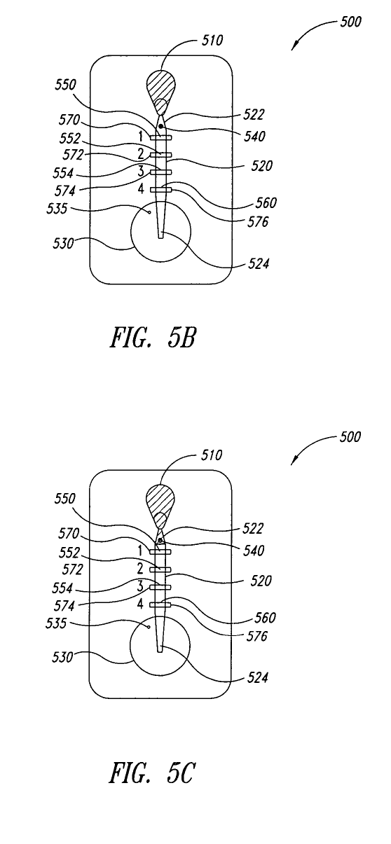

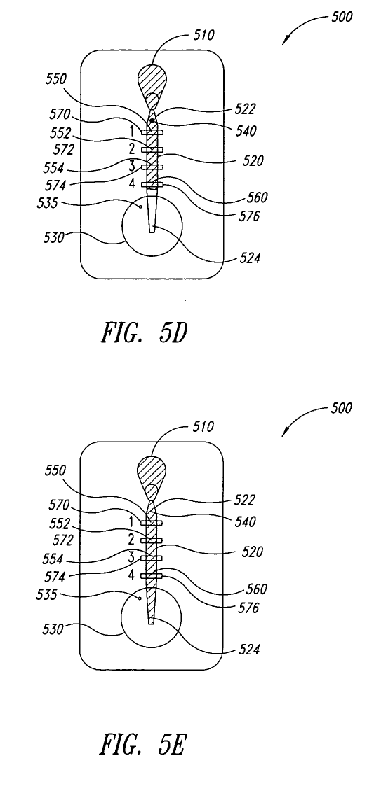

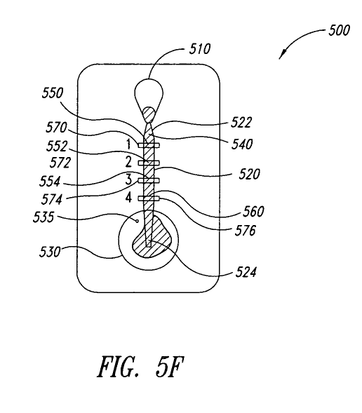

FIGS. 1A-1F are a series of cross-sectional views illustrating the operation of a first embodiment of a microfluidic laminar flow detection strip device 100 in accordance with aspects of the present invention. As shown in FIG. 1A, device 100 comprises a first inlet 110 (for receiving a liquid sample), a microfluidic channel 120 having a first end 122 and a second end 124, wherein first end 122 is fluidly connected to first inlet 110, and a bellows pump 130 fluidly connected to second end 124 of microfluidic channel 120. Microfluidic channel 120 may be straight, as illustrated in FIGS. 5A-5F, or may have a serpentine shape as illustrated in FIG. 1A to provide a longer reaction channel. Bellows pump 130 comprises an absorbent material (not specifically shown), such as cotton, disposed therein. In addition, in the embodiment of FIG. 1A, bellows pump 130 comprises a vent hole 135.

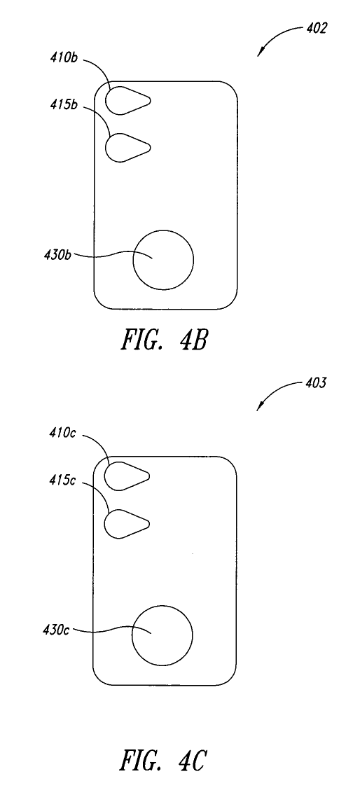

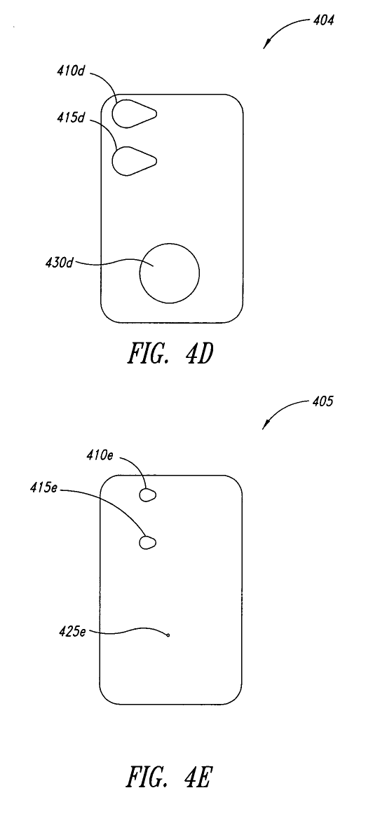

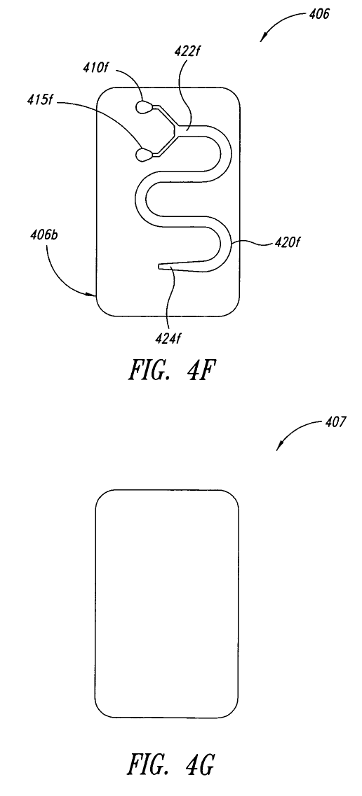

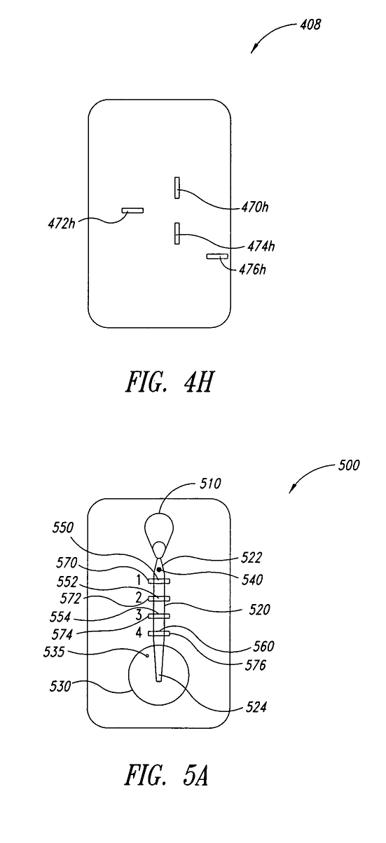

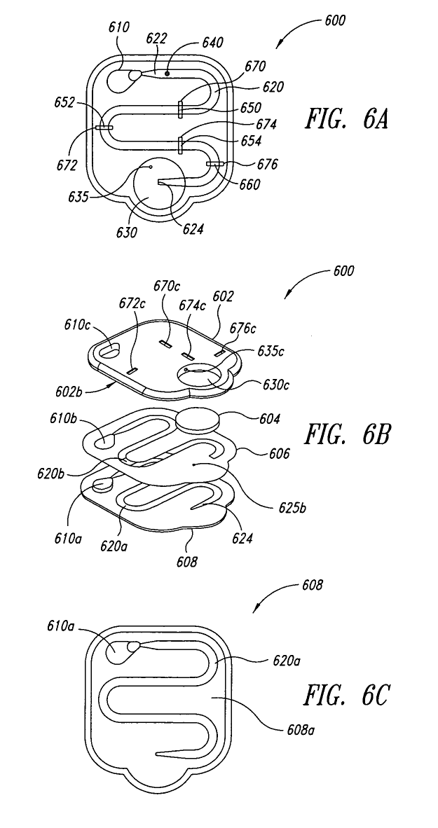

As illustrated, device 100 is in the form of a cartridge, however, the form of device 100 is not essential to the present invention, and persons of ordinary skill in the art can readily select a suitable form for a given application. Furthermore, as described in more detail with respect to FIGS. 4A-4I and 6A-6C, the microfluidic devices of the present invention, such as device 100, may be constructed from a material, such as transparent plastic, mylar, or latex, using a method such as injection molding or lamination.

As further shown in FIG. 1A, device 100 comprises a dried reagent zone 140 within microfluidic channel 120. Dried reagent zone 140 comprises a first reagent (not specifically shown) and a control reagent (not specifically shown) printed thereon. The first reagent comprises a first detection antibody (not specifically shown) conjugated to a dyed substrate bead (not specifically shown) or functionalized for calorimetric development, and the control reagent comprises a control detection antibody (not specifically shown) conjugated to a dyed substrate bead (not specifically shown) or functionalized for colorimetric development. The first detection antibody is specific to a particular analyte (e.g., antigen) of interest. Representative detection antibodies include, but are not limited to antibodies to antigens, such as infection agents (e.g., influenza, E. coli, etc. . . . ). An example of a representative dyed substrate bead is a dyed streptavidin microparticle. An example of a representative antibody functionalized for colorimetric analysis is poly-HRP-SA-40. The control detection antibody is not specific for a particular analyte and is included to control for nonspecific reactivity (negative control) or a positive control. Representative control detection antibodies include (but are not limited to) antibodies to normal flora (e.g., E. coli in feces). The first reagent and control reagent are printed onto microfluidic channel 120 such that the antibody/bead conjugates or functionalized antibodies are capable of being transported by a liquid sample though microfluidic channel 120.

In device 100 of FIG. 1A, dried reagent zone 140 further comprises a second reagent (not specifically shown) and a third reagent (not specifically shown). Each of the second and third reagents comprise a detection antibody (not specifically shown) conjugated to a dyed substrate bead (not specifically shown) or functionalized for calorimetric development. The second detection antibody is specific to a second analyte (e.g., antigen) of interest and the third detection antibody is specific to a third analyte (e.g., antigen) of interest. As one of skill in the art will appreciate, dried reagent zone may comprise as many (or as few) reagents as there are analytes of interest (in addition to the control reagent). For example, if there is only one analyte of interest, dried reagent zone 140 will only comprise a first reagent and a control reagent. Similarly, if there are five analytes of interest, dried reagent zone 140 will comprise first, second, third, fourth and fifth reagents, in addition to the control reagent.

As further shown in FIG. 1A, device 100 comprises a first bound antibody zone 150 within microfluidic channel 120 having a first bound antibody (not specifically shown) printed thereon, a second bound antibody zone 152 within microfluidic channel 120 having a second bound antibody (not specifically shown) printed thereon, and a third bound antibody zone 154 within microfluidic channel 120 having a third bound antibody (not specifically shown) printed thereon. The first, second and third bound antibodies are specific to the first, second and third analytes of interest, and may the same as, or different than, the first, second and third detection antibodies. The first, second and third bound antibodies are printed onto microfluidic channel 120 in first, second and third bound antibody zones 150, 152, 154 such that the antibodies are immobilized and are not capable of being transported by a liquid sample though microfluidic channel 120. As one of skill in the art will appreciate, device 100 may comprise as many (or as few) bound antibody zones as there are analytes of interest. For example, if there is only one analyte of interest, device 100 will only comprise a first bound antibody zone. Similarly, if there are five analytes of interest, device 100 will comprise first, second, third, fourth and fifth bound antibody zones.

As further shown in FIG. 1A, device 100 comprises a control zone 160 within microfluidic channel 120 having a control bound antibody (not specifically shown) printed thereon. Similar to first, second and third bound antibody zones 150, 152, 154, the control bound antibody is printed onto microfluidic channel 120 in control zone 160 such that the control bound antibody is immobilized and is not capable of being transported by a liquid sample through microfluidic channel 120. The control bound antibody may be the same as, or different than, the control detection antibody.

As one of ordinary skill in the art will appreciate, all of the foregoing reagents and antibodies may be printed onto microfluidic channel 120 during the manufacture of device 100 by methods such as ink jet printing, micro drop printing and transfer printing. Further, in order to ensure that the antibodies in bound antibody zones 150, 152, 154 and control zone 160 are immobilized, the surface of microfluidic channel 120 may be plasma treated prior to printing. Such plasma treatment is defined as low pressure oxygen plasma (or could be replaced with carbon dioxide, argon or mixtures of gases) directed to plastic surface for modifying the surface chemistry plastic surface. In addition, in order to ensure that indiscriminate binding of the reagents and antibodies to microfluidic channel 120 does not occur during periods of fluid flow within microfluidic channel 120, a blocking solution (such as casein or bovine serum albumin) may be flowed through microfluidic channel 120 during manufacture of device 100. Such a blocking solution prevents nonspecific binding within the channel.

During operation of device 100, a liquid sample is placed into first inlet 110 (as shown in FIG. 1B), bellows pump 130 is depressed, either manually by a user or mechanically by an external device, vent hole 135 is substantially sealed, such as by covering vent hole 135 with a user's finger, and bellows pump 130 is then released. During depression of bellows pump 130, vent hole 135 remains uncovered so that fluid in bellows pump 130 may be expelled through vent hole 135. Upon release of bellows pump 130, a negative fluid pressure is created in microfluidic channel 120 and the liquid sample is drawn through, microfluidic channel 120 and into the absorbent material disposed in bellows pump 130 (as shown in FIGS. 1C-1F) by capillary forces.

Second end 124 of microfluidic channel 120 is sized to control the flow rate of the liquid sample through microfluidic channel 120. In this regard, in certain embodiments, the diameter of second end 124 is 25-500 μm, and, in more specific embodiments, the diameter of second end 124 is 50-100 μm. Microfluidic channel 120 is typically 2,000-10,000 μm wide, more typically 3,000-6,000 μm wide, and 10-500 μm high, more typically 50-150 μm high.

As the liquid sample is drawn through microfluidic channel 120, the liquid sample hydrates dried reagent zone 140 and the first, second, third and control reagents are transported by the liquid sample though microfluidic channel 120. While in solution in the liquid sample, the first, second, third and control detection antibodies interact with (i.e., bind to) any corresponding analytes (e.g., antigens) of interest present in the liquid sample. Subsequently, as the liquid sample passes over first, second and third bound antibody zones 150, 152 and 154, if any corresponding analytes of interest are present in the liquid sample, such analytes (as well as the antibody/bead conjugates or functionalized antibodies to which such analytes are bound) will bind to, and become immobilized on, first, second and third bound antibody zones 150, 152 and 154. Similarly, as the liquid sample passes over control zone 160, the corresponding analyte present in the liquid sample (as well as the antibody/bead conjugates or functionalized antibodies to which such analyte is bound) will bind to, and become immobilized on, control zone 160.

As shown in FIG. 1A, device 100 may comprise optical viewing windows 170, 172, 174, 176 positioned over first, second and third bound antibody zones 150, 152, 154 and control zone 160, respectively. As shown in FIG. 1A, optical viewing windows 170, 172, 174, 176 may be labeled with, e.g., numbers and/or letters to facilitate identification of the zones. If dyed substrate beads are utilized in device 100, visual inspection of device 100 can be used to ascertain whether a particular analyte of interest was present in the liquid sample by determining whether any color change has occurred in the corresponding bound antibody zone. Similarly, if antibodies functionalized for colorimetric development are utilized in device 100, a developing solution (e.g., 3,3′,5,5′-tetramehtyl benzidine (TMB)) is flowed through microfluidic channel 120 following the liquid sample and prior to visual inspection for color changes. As one of skill in the art will appreciate, a color change in control zone 160 indicates that the liquid sample has indeed hydrated dried reagent zone 140 and flowed through microfluidic channel 120 as desired.

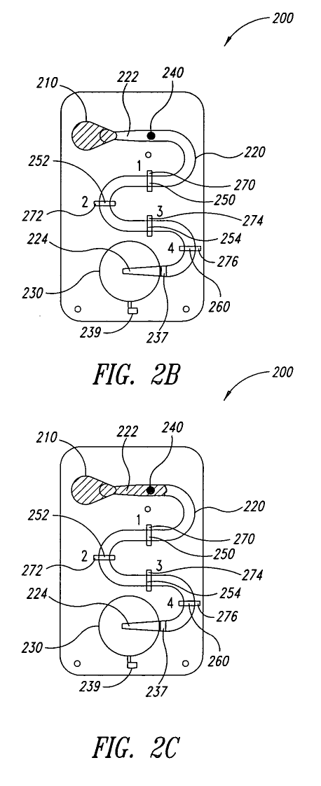

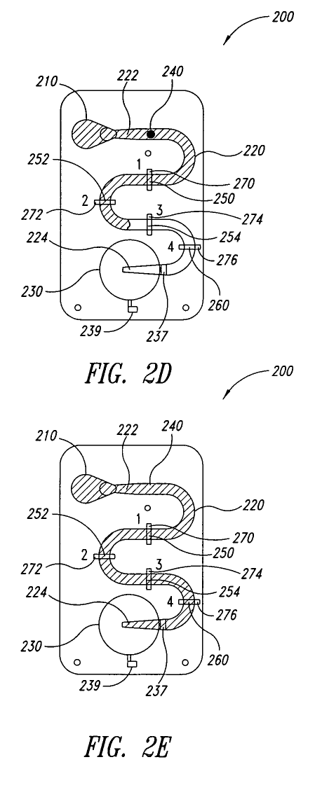

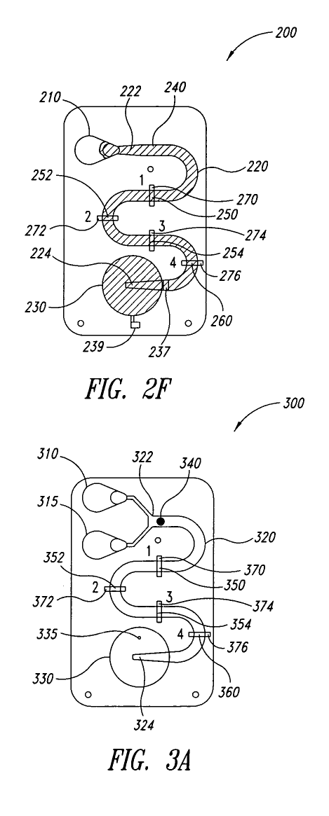

FIGS. 2A-2F are a series of cross-sectional views illustrating the operation of a second embodiment of a microfluidic laminar flow detection strip device 200 in accordance with aspects of the present invention. As shown in FIG. 2A, device 200 is similar to device 100 of FIG. 1A and comprises a first inlet 210 (for receiving a liquid sample), a microfluidic channel 220 having a first end 222 and a second end 224, wherein first end 222 is fluidly connected to first inlet 210, and a bellows pump 230 fluidly connected to second end 224 of microfluidic channel 220. Microfluidic channel 220 may be straight, as illustrated in FIGS. 5A-5F, or may have a serpentine shape as illustrated in FIG. 2A to provide a longer reaction channel. As in device 100 of FIG. 1A, bellows pump 230 comprises an absorbent material (not specifically shown) disposed therein.

Rather than providing a vent hole in bellows pump 230 as in FIG. 1A, device 200 utilizes first and second check valves, 237 and 239, respectively, to prevent the fluid in bellows pump 230 from being expelled into microfluidic channel 220 during depression of bellows pump 230. Check valves, also known as one-way valves, permit fluid flow in one direction only. Exemplary check valves for use in microfluidic structures are described in U.S. Pat. No. 6,431,212, which is hereby incorporated by reference in its entirety. First check valve 237 is fluidly connected to bellows pump 230 and permits fluid flow from microfluidic channel 220 into bellows pump 230 and prevents fluid flow from bellows pump 230 into microfluidic channel 220. Second check valve 239 is fluidly connected to bellows pump 230 and permits fluid flow away from the bellows pump (e.g., by venting to the atmosphere).

As illustrated, device 200 is in the form of a cartridge, however, the form of device 200 is not essential to the present invention, and persons of ordinary skill in the art can readily select a suitable form for a given application. Furthermore, as described in more detail with respect to FIGS. 4A-4I and 6A-6C, the microfluidic devices of the present invention, such as device 200, may be constructed from a material, such as transparent plastic, mylar, or latex, using a method such as injection molding or lamination.

As in device 100 of FIG. 1A, and as further shown in FIG. 2A, device 200 comprises a dried reagent zone 240 within microfluidic channel 220. Dried reagent zone 240 comprises a first reagent (not specifically shown) and a control reagent (not specifically shown) printed thereon. The first reagent comprises a first detection antibody (not specifically shown) conjugated to a dyed substrate bead (not specifically shown) or functionalized for colorimetric development, and the control reagent comprises a control detection antibody (not specifically shown) conjugated to a dyed substrate bead (not specifically shown) or functionalized for colorimetric development. The first reagent and control reagent are printed onto microfluidic channel 220 such that the antibody/bead conjugates or functionalized antibodies are capable of being transported by a liquid sample though microfluidic channel 220.

In device 200 of FIG. 2A, dried reagent zone 240 further comprises a second reagent (not specifically shown) and a third reagent (not specifically shown). Each of the second and third reagents comprise a detection antibody (not specifically shown) conjugated to a dyed substrate bead (not specifically shown) or functionalized for calorimetric development. As one of skill in the art will appreciate, dried reagent zone may comprise as many (or as few) reagents as there are analytes of interest (in addition to the control reagent). For example, if there is only one analyte of interest, dried reagent zone 240 will only comprise a first reagent and a control reagent. Similarly, if there are five analytes of interest, dried reagent zone 240 will comprise first, second, third, fourth and fifth reagents, in addition to the control reagent.

As further shown in FIG. 2A, device 200 comprises a first bound antibody zone 250 within microfluidic channel 220 having a first bound antibody (not specifically shown) printed thereon, a second bound antibody zone 252 within microfluidic channel 220 having a second bound antibody (not specifically shown) printed thereon, and a third bound antibody zone 254 within microfluidic channel 220 having a third bound antibody (not specifically shown) printed thereon. The first, second and third bound antibodies are printed onto microfluidic channel 220 in first, second and third bound antibody zones 250, 252, 254 such that the antibodies are immobilized and are not capable of being transported by a liquid sample though microfluidic channel 220. As one of skill in the art will appreciate, device 200 may comprise as many (or as few) bound antibody zones as there are analytes of interest. For example, if there is only one analyte of interest, device 200 will only comprise a first bound antibody zone. Similarly, if there are five analytes of interest, device 200 will comprise first, second, third, fourth and fifth bound antibody zones.

As further shown in FIG. 2A, device 200 comprises a control zone 260 within microfluidic channel 220 having a control bound antibody (not specifically shown) printed thereon. Similar to first, second and third bound antibody zones 250, 252, 254, the control bound antibody is printed onto microfluidic channel 220 in control zone 260 such that the control bound antibody is immobilized and is not capable of being transported by a liquid sample through microfluidic channel 220.

As one of ordinary skill in the art will appreciate, as in device 100 of FIG. 1A, all of the foregoing reagents and antibodies may be printed onto microfluidic channel 220 during the manufacture of device 200 by methods such as ink jet printing, micro drop printing and transfer printing. Further, in order to ensure that the antibodies in bound antibody zones 250, 252, 254 and control zone 260 are immobilized, the surface of microfluidic channel 220 may be plasma treated prior to printing. In addition, in order to ensure that indiscriminate binding of the reagents and antibodies to microfluidic channel 220 does not occur during periods of fluid flow within microfluidic channel 220, a blocking solution may be flowed through microfluidic channel 220 during manufacture of device 200.

During operation of device 200, a liquid sample is placed into first inlet 210 (as shown in FIG. 2B), bellows pump 230 is depressed, either manually by a user or mechanically by an external device, and bellows pump 230 is then released. During depression of bellows pump 230, first check valve 237 remains closed and prevents fluid flow from bellows chamber 230 into microfluidic channel 220; second check valve 239 opens and expels the fluid displaced from bellows pump 230. Upon release of bellows pump 230, a negative fluid pressure is created in microfluidic channel 220, first check valve 237 opens and permits fluid flow from microfluidic channel 220 into bellows pump 230, second check valve 239 closes and prevents fluid flow into bellows pump 230 from, e.g., the atmosphere, and the liquid sample is drawn through, microfluidic channel 220 and into the absorbent material disposed in bellows pump 230 (as shown in FIGS. 2C-2F) by capillary forces.

Second end 224 of microfluidic channel 220 is sized to control the flow rate of the liquid sample through microfluidic channel 220. In this regard, in certain embodiments, the diameter of second end 224 is 25-500 μm, and, in more specific embodiments, the diameter of second end 224 is 50-100 μm. Microfluidic channel 220 is typically 2,000-10,000 μm wide, more typically 3,000-6,000 μm wide, and 10-500 μm high, more typically 50-150 μm high.

As the liquid sample is drawn through microfluidic channel 220, the liquid sample hydrates dried reagent zone 240 and the first, second, third and control reagents are transported by the liquid sample though microfluidic channel 220. While in solution in the liquid sample, the first, second, third and control detection antibodies interact with (i.e., bind to) any corresponding analytes (e.g., antigens) of interest present in the liquid sample. Subsequently, as the liquid sample passes over first, second and third bound antibody zones 250, 252 and 254, if any corresponding analytes of interest are present in the liquid sample, such analytes (as well as the antibody/bead conjugates or functionalized antibodies to which such analytes are bound) will bind to, and become immobilized on, first, second and third bound antibody zones 250, 252 and 254. Similarly, as the liquid sample passes over control zone 260, the corresponding analyte present in the liquid sample (as well as the antibody/bead conjugates or functionalized antibodies to which such analyte is bound) will bind to, and become immobilized on, control zone 260.

As shown in FIG. 2A, device 200 may comprise optical viewing windows 270, 272, 274, 276 positioned over first, second and third bound antibody zones 250, 252, 254 and control zone 260, respectively. As shown in FIG. 2A, optical viewing windows 270, 272, 274, 276 may be labeled with, e.g., numbers and/or letters to facilitate identification of the zones. If dyed substrate beads are utilized in device 200 are dyed, visual inspection of device 200 can be used to ascertain whether a particular analyte of interest was present in the liquid sample by determining whether any color change has occurred in the corresponding bound antibody zone. Similarly, if antibodies functionalized for colorimetric development are utilized in device 200, a developing solution (e.g., TMB) is flowed through microfluidic channel 220 following the liquid sample and prior to visual inspection for color changes. As one of skill in the art will appreciate, a color change in control zone 260 indicates that the liquid sample has indeed hydrated dried reagent zone 240 and flowed through microfluidic channel 220 as desired.

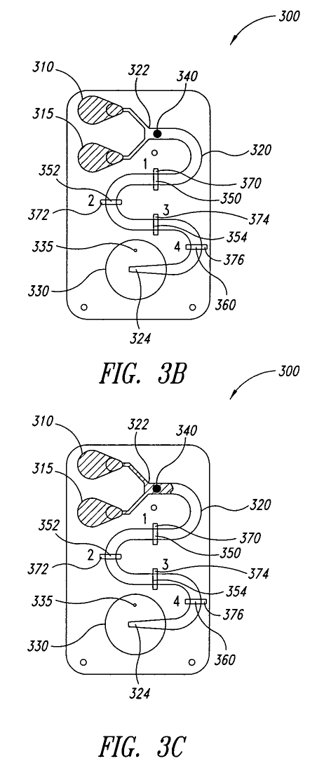

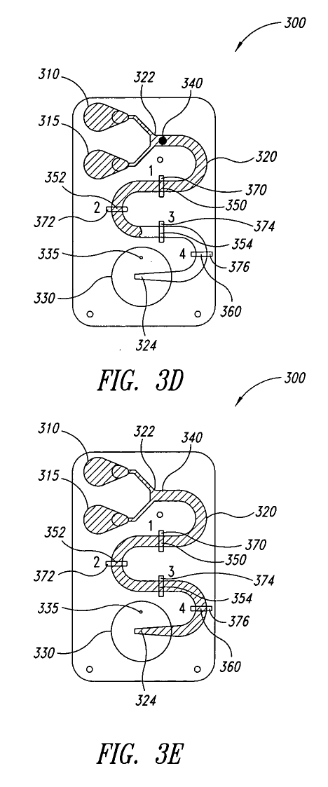

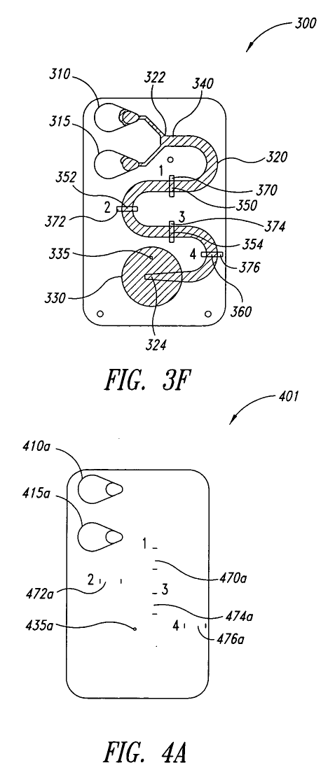

FIGS. 3A-3F are a series of cross-sectional views illustrating the operation of a third embodiment of a microfluidic laminar flow detection strip device in accordance with aspects of the present invention. As shown in FIG. 3A, device 300 is similar to device 100 of FIG. 1A and comprises a first inlet 310 (for receiving a liquid sample), a microfluidic channel 320 having a first end 322 and a second end 324, wherein first end 322 is fluidly connected to first inlet 310, and a bellows pump 330 fluidly connected to second end 324 of microfluidic channel 320. Microfluidic channel 320 may be straight, as illustrated in FIGS. 5A-5F, or may have a serpentine shape as illustrated in FIG. 3A to provide a longer reaction channel. As in device 100 of FIG. 1A, bellows pump 330 comprises an absorbent material (not specifically shown) disposed therein. In addition, in the embodiment of FIG. 1A, bellows pump 330 comprises a vent hole 335.

In addition, as shown in FIG. 3A, device 300 further comprises a second inlet 315 (for receiving a liquid sample) fluidly connected to first end 322 of microfluidic channel 320. By providing a second inlet 315, device 300 permits two different liquid samples to be introduced into microfluidic channel 320 in parallel laminar flow. Such an embodiment may be advantageous if diffusion of certain particles between the two fluid streams is desired. Alternatively, a single liquid sample may be introduced into both first and second inlets 310, 315.

As illustrated, device 300 is in the form of a cartridge, however, the form of device 300 is not essential to the present invention, and persons of ordinary skill in the art can readily select a suitable form for a given application. Furthermore, as described in more detail with respect to FIGS. 4A-41 and 6A-6C, the microfluidic devices of the present invention, such as device 300, may be constructed from a material, such as transparent plastic, mylar, or latex, using a method such as injection molding or lamination.

As in device 100 of FIG. 1A, and as further shown in FIG. 3A, device 300 comprises a dried reagent zone 340 within microfluidic channel 320. Dried reagent zone 340 comprises a first reagent (not specifically shown) and a control reagent (not specifically shown) printed thereon. The first reagent comprises a first detection antibody (not specifically shown) conjugated to a dyed substrate bead (not specifically shown) or functionalized for colorimetric development, and the control reagent comprises a control detection antibody (not specifically shown) conjugated to a dyed substrate bead (not specifically shown) or functionalized for colorimetric development. The first reagent and control reagent are printed onto microfluidic channel 320 such that the antibody/bead conjugates or functionalized antibodies are capable of being transported by a liquid sample though microfluidic channel 320.

In device 300 of FIG. 3A, dried reagent zone 340 further comprises a second reagent (not specifically shown) and a third reagent (not specifically shown). Each of the second and third reagents comprise a detection antibody (not specifically shown) conjugated to a dyed substrate bead (not specifically shown) or functionalized for colorimetric development. As one of skill in the art will appreciate, dried reagent zone may comprise as many (or as few) reagents as there are analytes of interest (in addition to the control reagent). For example, if there is only one analyte of interest, dried reagent zone 340 will only comprise a first reagent and a control reagent. Similarly, if there are five analytes of interest, dried reagent zone 340 will comprise first, second, third, fourth and fifth reagents, in addition to the control reagent.

As further shown in FIG. 3A, device 300 comprises a first bound antibody zone 350 within microfluidic channel 320 having a first bound antibody (not specifically shown) printed thereon, a second bound antibody zone 352 within microfluidic channel 320 having a second bound antibody (not specifically shown) printed thereon, and a third bound antibody zone 354 within microfluidic channel 320 having a third bound antibody (not specifically shown) printed thereon. The first, second and third bound antibodies are printed onto microfluidic channel 320 in first, second and third bound antibody zones 350, 352, 354 such that the antibodies are immobilized and are not capable of being transported by a liquid sample though microfluidic channel 320. As one of skill in the art will appreciate, device 300 may comprise as many (or as few) bound antibody zones as there are analytes of interest. For example, if there is only one analyte of interest, device 300 will only comprise a first bound antibody zone. Similarly, if there are five analytes of interest, device 300 will comprise first, second, third, fourth and fifth bound antibody zones.

As further shown in FIG. 3A, device 300 comprises a control zone 360 within microfluidic channel 320 having a control bound antibody (not specifically shown) printed thereon. Similar to first, second and third bound antibody zones 350, 352, 354, the control bound antibody is printed onto microfluidic channel 320 in control zone 360 such that the control bound antibody is immobilized and is not capable of being transported by a liquid sample through microfluidic channel 320.

As one of ordinary skill in the art will appreciate, as in device 100 of FIG. 1A, all of the foregoing reagents and antibodies may be printed onto microfluidic channel 320 during the manufacture of device 300 by methods such as ink jet printing, micro drop printing and transfer printing. Further, in order to ensure that the antibodies in bound antibody zones 350, 352, 354 and control zone 360 are immobilized, the surface of microfluidic channel 320 may be plasma treated prior to printing. In addition, in order to ensure that indiscriminate binding of the reagents and antibodies to microfluidic channel 320 does not occur during periods of fluid flow within microfluidic channel 320, a blocking solution may be flowed through microfluidic channel 320 during manufacture of device 300.

During operation of device 300, one or more liquid samples are placed into first and second inlets 310 and 315 (as shown in FIG. 3B), bellows pump 330 is depressed, either manually by a user or mechanically by an external device, vent hole 335 is substantially sealed, such as by covering vent hole 335 with a user's finger, and bellows pump 330 is then released. During depression of bellows pump 330, vent hole 335 remains uncovered so that fluid in bellows pump 330 may be expelled through vent hole 335. Upon release of bellows pump 330, a negative fluid pressure is created in microfluidic channel 320 and the liquid sample is drawn through, microfluidic channel 320 and into the absorbent material disposed in bellows pump 330 (as shown in FIGS. 3C-3F) by capillary forces.

Second end 324 of microfluidic channel 320 is sized to control the flow rate of the liquid sample through microfluidic channel 320. In this regard, in certain embodiments, the diameter of second end 324 is 25-500 μm, and, in more specific embodiments, the diameter of second end 324 is 50-100 μm. Microfluidic channel 320 is typically 2,000-10,000 μm wide, more typically 3,000-6,000 μm wide, and 10-500 μm high, more typically 50-150 μm high.

As the liquid sample is drawn through microfluidic channel 320, the liquid sample hydrates dried reagent zone 340 and the first, second, third and control reagents are transported by the liquid sample though microfluidic channel 320. While in solution in the liquid sample, the first, second, third and control detection antibodies interact with (i.e., bind to) any corresponding analytes (e.g., antigens) of interest present in the liquid sample. Subsequently, as the liquid sample passes over first, second and third bound antibody zones 350, 352 and 354, if any corresponding analytes of interest are present in the liquid sample, such analytes (as well as the antibodylbead conjugates or functionalized antibodies to which such analytes are bound) will bind to, and become immobilized on, first, second and third bound antibody zones 350, 352 and 354. Similarly, as the liquid sample passes over control zone 360, the corresponding analyte present in the liquid sample (as well as the antibody/bead conjugates or the functionalized antibodies to which such analyte is bound) will bind to, and become immobilized on, control zone 360.

As shown in FIG. 3A, device 300 may comprise optical viewing windows 370, 372, 374, 376 positioned over first, second and third bound antibody zones 350, 352, 354 and control zone 360, respectively. As shown in FIG. 3A, optical viewing windows 370, 372, 374, 376 may be labeled with, e.g., numbers and/or letters to facilitate identification of the zones. If dyed substrate beads are utilized in device 300, visual inspection of device 300 can be used to ascertain whether a particular analyte of interest was present in the liquid sample by determining whether any color change has occurred in the corresponding bound antibody zone. Similarly, if antibodies functionalized for calorimetric development are utilized in device 300, a developing solution (e.g., TMB) is flowed through microfluidic channel 320 following the liquid sample and prior to visual inspection for color changes. As one of skill in the art will appreciate, a color change in control zone 360 indicates that the liquid sample has indeed hydrated dried reagent zone 340 and flowed through microfluidic channel 320 as desired.

As shown in FIGS. 4A-41, a microfluidic laminar flow detection strip device similar to device 300 of FIGS. 3A-3F may be made from a plurality (e.g., seven in the illustrated embodiment) of individual laminate layers which are laminated together.

FIG. 4A shows the first (or top) laminate layer 401 which comprises (a) a first inlet cutout 410a extending through first laminate layer 401, (b) a second inlet cutout 415a extending through first laminate layer 401, (c) a vent hole 435a extending through first laminate layer 401, and (d) optical viewing windows 470a, 472a, 474a, 476a. As shown, optical viewing windows 470a, 472a, 474a, 476a may be labeled with, e.g., numbers and/or letters to facilitate identification of the corresponding bound antibody and control zones.

FIGS. 4B, 4C and 4D show the second, third and fourth laminate layers 402, 403, 404, each of which comprise (a) a first inlet cutout 410b, 410c, 410d, respectively, extending through second, third and fourth laminate layers 402, 403, 404, respectively, (b) a second inlet cutout 415b, 415c, 415d, respectively, extending through second, third and fourth laminate layers 402, 403, 404, respectively, and (c) a bellows pump cutout 430b, 430c, 430d, respectively, extending through second, third and fourth laminate layers 402, 403, 404, respectively.

FIG. 4E shows the fifth laminate layer 405 which comprises (a) a first inlet cutout 410e extending through fifth laminate layer 405, (b) a second inlet cutout 415e extending through fifth laminate layer 405, and (c) a through-hole 425e extending through fifth laminate layer 405. Through-hole 425b fluidly connects second end 424f of microfluidic channel cutout 420f in sixth laminate layer 406 and bellows pump cutout 430d in fourth laminate layer 404. Through-hole 425b is sized to control the flow rate of the liquid sample through the microfluidic channel formed by the assembly of fifth, sixth and seventh laminate layers 405, 406, 407. In this regard, in certain embodiments, the diameter of through-hole 425b is 25-500 μm, and, in more specific embodiments, the diameter of through-hole 425b is 50-100 μm.

FIG. 4F shows the sixth laminate layer 406 which comprises (a) a first inlet cutout 410f extending through sixth laminate layer 406, (b) a second inlet cutout 415f extending through sixth laminate layer 406, and (c) a microfluidic channel cutout 420f, having a first end 422f and a second end 424f, extending through sixth laminate layer 406.

FIG. 4G shows the seventh (or bottom) laminate layer 407 which merely comprises a solid layer.

First, second, third and control reagents, first, second and third bound antibodies and the control bound antibody are printed onto the surface of seventh laminate layer 407 during the manufacture of the device by methods such as ink jet printing, micro drop printing and transfer printing. The first, second, third and control reagents are printed such that the antibody/bead conjugates or functionalized antibodies thereof are capable of being transported by a liquid sample though the microfluidic channel formed by the assembly of fifth, sixth and seventh laminate layers 405, 406, 407. The first, second and third and control bound antibodies are printed such that the antibodies are immobilized and are not capable of being transported by a liquid sample though such microfluidic channel. As discussed previously, in order to ensure that the antibodies in the bound antibody zones and the control zone are immobilized, the surface of seventh laminate layer 407 may be plasma treated prior to printing. To ensure that only the portions of seventh laminate layer 407 representing the bound antibody zones and the control zone are plasma treated, a masking layer 408 (shown in FIG. 4H) may be placed on top of seventh laminate layer 407. As shown, masking layer 408 comprises cutouts 470h, 472h, 474h and 476h overlaying the bound antibody zones and the control zone. In addition, in order to ensure that indiscriminate binding of the reagents and antibodies to the microfluidic channel formed by the assembly of fifth, sixth and seventh laminate layers 405, 406, 407 does not occur during periods of fluid flow within such microfluidic channel, a blocking solution may be flowed through such microfluidic channel during manufacture of the device.

As one of ordinary skill in the art will appreciate, when the foregoing laminate layers are laminated together, a microfluidic laminar flow detection strip device similar to device 300 of FIGS. 3A-3F will be produced.

FIGS. 5A-5F are a series of cross-sectional views illustrating the operation of a fourth embodiment of a microfluidic laminar flow detection strip device 500 in accordance with aspects of the present invention. As shown in FIG. 5A, device 500 is similar to device 100 of FIG. 1A and comprises a first inlet 510 (for receiving a liquid sample), a microfluidic channel 520 having a first end 522 and a second end 524, wherein first end 522 is fluidly connected to first inlet 510, and a bellows pump 530 fluidly connected to second end 524 of microfluidic channel 520. Unlike microfluidic channel 120 of FIG. 1A, microfluidic channel 520 is straight. As in device 100 of FIG. 1A, bellows pump 530 comprises an absorbent material (not specifically shown) disposed therein. In addition, in the embodiment of FIG. 5A, bellows pump 530 comprises a vent hole 535.

As illustrated, device 500 is in the form of a cartridge, however, the form of device 500 is not essential to the present invention, and persons of ordinary skill in the art can readily select a suitable form for a given application. Furthermore, as described in more detail with respect to FIGS. 4A-4I and 6A-6C, the microfluidic devices of the present invention, such as device 500, may be constructed from a material, such as transparent plastic, mylar, or latex, using a method such as injection molding or lamination.

As in device 100 of FIG. 1A, and as further shown in FIG. 5A, device 500 comprises a dried reagent zone 540 within microfluidic channel 520. Dried reagent zone 540 comprises a first reagent (not specifically shown) and a control reagent (not specifically shown) printed thereon. The first reagent comprises a first detection antibody (not specifically shown) conjugated to a dyed substrate bead (not specifically shown) or functionalized for colorimetric development, and the control reagent comprises a control detection antibody (not specifically shown) conjugated to a dyed substrate bead (not specifically shown) or functionalized for colorimetric development. The first reagent and control reagent are printed onto microfluidic channel 520 such that the antibody/bead conjugates or functionalized antibodies are capable of being transported by a liquid sample though microfluidic channel 520.

In device 500 of FIG. 5A, dried reagent zone 540 further comprises a second reagent (not specifically shown) and a third reagent (not specifically shown). Each of the second and third reagents comprise a detection antibody (not specifically shown) conjugated to a dyed substrate bead (not specifically shown) or functionalized for colorimetric development. As one of skill in the art will appreciate, dried reagent zone may comprise as many (or as few) reagents as there are analytes of interest (in addition to the control reagent). For example, if there is only one analyte of interest, dried reagent zone 540 will only comprise a first reagent and a control reagent. Similarly, if there are five analytes of interest, dried reagent zone 540 will comprise first, second, third, fourth and fifth reagents, in addition to the control reagent.

As further shown in FIG. 5A, device 500 comprises a first bound antibody zone 550 within microfluidic channel 520 having a first bound antibody (not specifically shown) printed thereon, a second bound antibody zone 552 within microfluidic channel 520 having a second bound antibody (not specifically shown) printed thereon, and a third bound antibody zone 554 within microfluidic channel 520 having a third bound antibody (not specifically shown) printed thereon. The first, second and third bound antibodies are printed onto microfluidic channel 520 in first, second and third bound antibody zones 550, 552, 554 such that the antibodies are immobilized and are not capable of being transported by a liquid sample though microfluidic channel 520. As one of skill in the art will appreciate, device 500 may comprise as many (or as few) bound antibody zones as there are analytes of interest. For example, if there is only one analyte of interest, device 500 will only comprise a first bound antibody zone. Similarly, if there are five analytes of interest, device 500 will comprise first, second, third, fourth and fifth bound antibody zones.

As further shown in FIG. 5A, device 500 comprises a control zone 560 within microfluidic channel 520 having a control bound antibody (not specifically shown) printed thereon. Similar to first, second and third bound antibody zones 550, 552, 554, the control bound antibody is printed onto microfluidic channel 520 in control zone 560 such that the control bound antibody is immobilized and is not capable of being transported by a liquid sample through microfluidic channel 520.

As one of ordinary skill in the art will appreciate, all of the foregoing reagents and antibodies may be printed onto microfluidic channel 520 during the manufacture of device 500 by methods such as ink jet printing, micro drop printing and transfer printing. Further, in order to ensure that the antibodies in bound antibody zones 550, 552, 554 and control zone 560 are immobilized, the surface of microfluidic channel 520 may be plasma treated prior to printing. In addition, in order to ensure that indiscriminate binding of the reagents and antibodies to microfluidic channel 520 does not occur during periods of fluid flow within microfluidic channel 520, a blocking solution may be flowed through microfluidic channel 520 during manufacture of device 500.

During operation of device 500, a liquid sample is placed into first inlet 510 (as shown in FIG. 5B), bellows pump 530 is depressed, either manually by a user or mechanically by an external device, vent hole 535 is substantially sealed, such as by covering vent hole 535 with a user's finger, and bellows pump 530 is then released. During depression of bellows pump 530, vent hole 535 remains uncovered so that fluid in bellows pump 530 may be expelled through vent hole 535. Upon release of bellows pump 530, a negative fluid pressure is created in microfluidic channel 520 and the liquid sample is drawn through, microfluidic channel 520 and into the absorbent material disposed in bellows pump 530 (as shown in FIGS. 5C-5F) by capillary forces.

Second end 524 of microfluidic channel 520 is sized to control the flow rate of the liquid sample through microfluidic channel 520. In this regard, in certain embodiments, the diameter of second end 524 is 25-500 μm, and, in more specific embodiments, the diameter of second end 524 is 50-100 μm. Microfluidic channel 520 is typically 2,000-10,000 μm wide, more typically 3,000-6,000 μm wide, and 10-500 μm high, more typically 50-150 μm high.

As the liquid sample is drawn through microfluidic channel 520, the liquid sample hydrates dried reagent zone 540 and the first, second, third and control reagents are transported by the liquid sample though microfluidic channel 520. While in solution in the liquid sample, the first, second, third and control detection antibodies interact with (i.e., bind to) any corresponding analytes (e.g., antigens) of interest present in the liquid sample. Subsequently, as the liquid sample passes over first, second and third bound antibody zones 550, 552 and 554, if any corresponding analytes of interest are present in the liquid sample, such analytes (as well as the antibody/bead conjugates or functionalized antibodies to which such analytes are bound) will bind to, and become immobilized on, first, second and third bound antibody zones 550, 552 and 554. Similarly, as the liquid sample passes over control zone 560, the corresponding analyte present in the liquid sample (as well as the antibody/bead conjugates or functionalized antibodies to which such analyte is bound) will bind to, and become immobilized on, control zone 560.

As shown in FIG. 5A, device 500 may comprise optical viewing windows 570, 572, 574, 576 positioned over first, second and third bound antibody zones 550, 552, 554 and control zone 560, respectively. As shown in FIG. 5A, optical viewing windows 570, 572, 574, 576 may be labeled with, e.g., numbers and/or letters to facilitate identification of the zones. If dyed substrate beads are utilized in device 500, visual inspection of device 500 can be used to ascertain whether a particular analyte of interest was present in the liquid sample by determining whether any color change has occurred in the corresponding bound antibody zone. Similarly, if antibodies functionalized for colorimetric development are utilized in device 500, a developing solution (e.g., TMB) is flowed through microfluidic channel 520 following the liquid sample and prior to visual inspection for color changes. As one of skill in the art will appreciate, a color change in control zone 560 indicates that the liquid sample has indeed hydrated dried reagent zone 540 and flowed through microfluidic channel 520 as desired.

As shown in FIGS. 6A-6C, a microfluidic laminar flow detection strip device 600 similar to device 100 of FIGS. 1A-1F may be made from the assembly of two injection molded layers 602, 608 and an adhesive layer 606. As shown in FIG. 6A, device 600 comprises a first inlet 610, a microfluidic channel 620 having a first end 622 and a second end 624, wherein first end 622 is fluidly connected to first inlet 610, and a bellows pump 630 fluidly connected to second end 624 of microfluidic channel 620. Similar to device 100 of FIG. 1A, bellows pump 630 comprises an absorbent material (not specifically shown) and a vent hole 635.

As further shown in FIG. 6A, device 600 comprises a dried reagent zone 640 within microfluidic channel 620. Dried reagent zone 640 comprises a first reagent (not specifically shown), a second reagent (not specifically shown), a third reagent (not specifically shown) and a control reagent (not specifically shown) printed thereon. The first reagent comprises a first detection antibody (not specifically shown) conjugated to a dyed substrate bead (not specifically shown) or functionalized for colorimetric development. The second reagent comprises a second detection antibody (not specifically shown) conjugated to a dyed substrate bead (not specifically shown) or functionalized for calorimetric development. The third reagent comprises a third detection antibody (not specifically shown) conjugated to a dyed substrate bead (not specifically shown) or functionalized for calorimetric development. The control reagent comprises a control detection antibody (not specifically shown) conjugated to a dyed substrate bead (not specifically shown) or functionalized for calorimetric development. Similar to the above embodiments, as one of skill in the art will appreciate, dried reagent zone may comprise as many (or as few) reagents as there are analytes of interest (in addition to the control reagent).

As further shown in FIG. 6A, device 600 comprises a first bound antibody zone 650 within microfluidic channel 620 having a first bound antibody (not specifically shown) printed thereon, a second bound antibody zone 652 within microfluidic channel 620 having a second bound antibody (not specifically shown) printed thereon, and a third bound antibody zone 654 within microfluidic channel 620 having a third bound antibody (not specifically shown) printed thereon. Again, as one of skill in the art will appreciate, device 600 may comprise as many (or as few) bound antibody zones as there are analytes of interest. In addition, as further shown in FIG. 6A, device 600 comprises a control zone 660 within microfluidic channel 620 having the control bound antibody (not specifically shown) printed thereon.

As shown in FIG. 6A, device 600 comprises optical viewing windows 670, 672, 674, 676 positioned over first, second and third bound antibody zones 650, 652, 654 and control zone 660, respectively.

As shown in FIG. 6B, device 600 is made from the assembly of top and bottom injection molded layers 602, 608 and middle adhesive layer 606. As shown in FIGS. 6B and 6C, bottom injection molded layer 608 comprises a first inlet recess 610a and a microfluidic channel recess 620a in the top surface 608a of bottom injection molded layer 608. As shown in FIG. 6B, middle adhesive layer 606 comprises a first inlet cutout 610b and a microfluidic channel cutout 620b extending through middle adhesive layer 606. In addition, middle adhesive layer 606 comprises a through-hole 625b extending through middle adhesive layer 606. Through-hole 625b fluidly connects second end 624 of microfluidic channel recess 620a in bottom injection molded layer 608 and the absorbent pad 604 disposed between middle adhesive layer 606 and top injection molded layer 602. Through-hole 625b is sized to control the flow rate of the liquid sample through the microfluidic channel formed by the assembly of top and bottom injection molded layers 602, 608 and middle adhesive layer 606. In this regard, in certain embodiments, the diameter of through-hole 625b is 25-500 μm, and, in more specific embodiments, the diameter of through-hole 625b is 50-100 μm. As shown in FIG. 6B, top injection molded layer 602 comprises a first inlet cutout 610c extending through top injection molded layer 602, a bellows pump recess 630c in the bottom surface 602b of top injection molded layer 602, a vent hole 635c in the portion of top injection molded layer 602 covering bellows pump recess 630c, and optical viewing windows 670c, 672c, 674c, 676c. As one of ordinary skill in the art will appreciate, the portion of top injection molded layer 602 covering bellows pump recess 630c must be flexible.

As one of ordinary skill in the art will appreciate, the first, second, third and control reagents, the first, second and third bound antibodies and the control bound antibody are printed into microfluidic channel recess 620a during the manufacture of device 600 by methods such as ink jet printing, micro drop printing and transfer printing. The first, second, third and control reagents are printed such that the antibody/bead conjugates or functionalized antibodies thereof are capable of being transported by a liquid sample though microfluidic channel 620. The first, second and third and control bound antibodies are printed such that the antibodies are immobilized and are not capable of being transported by a liquid sample though microfluidic channel 620. As discussed previously, in order to ensure that the antibodies in bound antibody zones 650, 652, 654 and control zone 660 are immobilized, the surface of microfluidic channel recess 620a may be plasma treated prior to printing. In addition, in order to ensure that indiscriminate binding of the reagents and antibodies to microfluidic channel 620 does not occur during periods of fluid flow within microfluidic channel 620, a blocking solution may be flowed through microfluidic channel 620 during manufacture of device 600.

When top and bottom injection molded layers 602, 608 and middle adhesive layer 606 are assembled as shown in FIG. 6B, (a) first inlet recess 610a and first inlet cutouts 610b and 610c cooperate to form first inlet 610, (b) microfluidic channel recess 620a, microfluidic channel cutout 620b and bottom surface 602b of top injection molded layer 602 cooperate to form microfluidic channel 620, and (c) middle adhesive layer 606 and bellows pump recess 630c cooperate to form bellows pump 630.

From the foregoing, and as set forth previously, it will be appreciated that, although specific embodiments of the invention have been described herein for purposes of illustration, various modifications may be made without deviating from the spirit and scope of the invention. For example, the disclosed microfluidic laminar flow detection strip devices may be utilized in combination with other sample preparation devices, and/or other qualitative or quantitative analysis devices. In addition, the disclosed microfluidic laminar flow detection strip devices may comprise addition microfluidic circuits for addition pre- or post-sample processing steps. Accordingly, the invention is not limited except as by the appended claims.

1. A microfluidic laminar flow detection strip device, comprising:
a first inlet;
a microfluidic channel having a first end and a second end, wherein the first end is fluidly connected to the first inlet;
a bellows pump fluidly connected to the second end of the microfluidic channel, wherein the bellows pump comprises an absorbent material disposed therein;
a dried reagent zone within the microfluidic channel, wherein the dried reagent zone comprises a first reagent and a control reagent printed thereon, the first reagent comprising a first detection antibody conjugated to a dyed substrate bead or functionalized for calorimetric development, and the control reagent comprising a control detection antibody conjugated to a dyed substrate bead or functionalized for colorimetric development;
a first bound antibody zone within the microfluidic channel, wherein the first bound antibody zone comprises a first bound antibody printed thereon; and
a control zone within the microfluidic channel, wherein the control zone comprises a control bound antibody printed thereon.

2. The microfluidic laminar flow detection strip device of claim 1, further comprising a second inlet fluidly connected to the first end of the microfluidic channel.

3. The microfluidic laminar flow detection strip device of claim 1, wherein:
the dried reagent zone further comprises a second reagent printed thereon, and the second reagent comprises a second detection antibody conjugated to a dyed substrate bead or functionalized for colorimetric development; and
the device further comprises a second bound antibody zone within the microfluidic channel, wherein the second bound antibody zone comprises a second bound antibody printed thereon.

4. The microfluidic laminar flow detection strip device of claim 3, wherein:
the dried reagent zone further comprises a third reagent printed thereon, and the third reagent comprises a third detection antibody conjugated to a dyed substrate bead or functionalized for colorimetric development; and
the device further comprises a third bound antibody zone within the microfluidic channel, wherein the third bound antibody zone comprises a third bound antibody printed thereon.

5. The microfluidic laminar flow detection strip device of claim 1 wherein the bellows pump further comprises a vent hole.

6. The microfluidic laminar flow detection strip device of claim 1, further comprising:
a first check valve fluidly connected to the bellows pump, wherein the first check valve permits fluid flow from the microfluidic channel into the bellows pump and prevents fluid flow from the bellows pump into the microfluidic channel; and
a second check valve fluidly connected to the bellows pump, wherein the second check valve permits fluid flow away from the bellows pump.

7. The microfluidic laminar flow detection strip device of claim 1 wherein the microfluidic channel has a serpentine shape.

8. The microfluidic laminar flow detection strip device of claim 1 wherein the second end of the microfluidic channel is sized to control fluid flow rate within the microfluidic channel.

9. The microfluidic laminar flow detection strip device of claim 8 wherein the second end of the microfluidic channel has a diameter of 25-500 μm.

10. The microfluidic laminar flow detection strip device of claim 9 wherein the second end of the microfluidic channel has a diameter of 50-100 μm.

11. The microfluidic laminar flow detection strip device of claim 1, further comprising optical viewing windows positioned over the first bound antibody zone and the control zone.

12. The microfluidic laminar flow detection strip device of claim 11 wherein the optical viewing windows are labeled.

13. The microfluidic laminar flow detection strip device of claim 1 wherein the first detection antibody is the same as the first bound antibody.

14. The microfluidic laminar flow detection strip device of claim 1 wherein the first detection antibody is different than the first bound antibody.

15. The microfluidic laminar flow detection strip device of claim 1 wherein the control detection antibody is the same as the control bound antibody.

16. The microfluidic laminar flow detection strip device of claim 1 wherein the control detection antibody is different than the control bound antibody.

17. The microfluidic laminar flow detection strip device of claim 1 wherein the device is formed from a plurality of laminate layers.

18. The mircofluidic laminar flow detection strip device of claim 1 wherein the device is formed from two injection molded layers and an adhesive layer.

19. A method of using the microfluidic laminar flow detection strip device of claim 1 to detect the presence of an analyte of interest in a liquid sample, the method comprising:
introducing the liquid sample into the first inlet of the device;
depressing the bellows pump;
releasing the bellows pump to draw the liquid sample through the microfluidic channel; and
visually inspecting the first bound antibody zone and the control zone for any color changes.

20. The method of claim 19 wherein:
the first reagent comprises a first detection antibody functionalized for calorimetric development;
the control reagent comprises a control detection antibody functionalized for calorimetric development; and
the method further comprises the following steps prior to the step of visually inspecting the first bound antibody zone and the control zone:
introducing a developing solution into the first inlet of the device;
depressing the bellows pump; and
releasing the bellows pump to draw the developing solution through the microfluidic channel.
